(19) United States
(12) Patent Application Publication
(10) Pub. No.: US 2013/0195725 A1
(43) Pub. Date: Aug. 1, 2013

(54) OZONATED LIQUID PRODUCTION AND DISTRIBUTION SYSTEMS

(71) Applicant: Daniel W. Lynn, Omaha, NE (US)

(72) Inventor: Daniel W. Lynn, Omaha, NE (US)

(73) Assignee: Food Safety Technology, LLC, Omaha, NE (US)

(21) Appl. No.: 13/800,057

(22) Filed: Mar. 13, 2013

Related U.S. Application Data
(63) Continuation-in-part of application No. 13/743,945, filed on Jan. 17, 2013.
Application No. 13/743,945 is a continuation-in-part of application No. 12/816,837, filed on Jun. 16, 2010.
Application No. 12/816,837 is a continuation-in-part of application No. 12/179,335, filed on Jul. 24, 2008.

(51) Int. Cl. B01J 19/08; A61L 2/20

(52) U.S. Cl. CPC B01J 19/087; A61L 2/202
USPC 422/111

(57) ABSTRACT

Ozonated liquid production and distribution system are described. The systems use multiple ozone gas generators to create ozone gas from ambient air. The ozone gas is injected into water or fluid by multiple injectors to form the ozonated liquid.

### CROSS REFERENCE TO RELATED APPLICATION

This application is a continuation-in-part of U.S. Nonprovisional patent application Ser. No. 13/743,945 filed Jan. 17, 2013, which is a continuation-in-part of U.S. Nonprovisional patent application Ser. No. 12/816,837 filed Jun. 16, 2010, which is a continuation-in-part of U.S. Nonprovisional patent application Ser. No. 12/179,335 filed Jul. 24, 2008, which are both hereby incorporated by reference.

### FIELD OF THE INVENTION

The present invention relates to ozonated liquid production and distribution systems that produce and distribute an ozonated liquid that may be used for cleaning and sanitizing applications and processes.

### BACKGROUND OF THE INVENTION

Ozone gas is unstable, which provides for it cleaning and sanitizing capabilities, but also makes consistent ozone levels difficult to maintain when the gas is mixed into a solution. Ozone gas cannot be packaged or stored and must be generated on site. The systems reduce the need for chemicals, hot water, and labor. Conventional cleaning systems often require the use of warm or hot water, which may form condensation in the surrounding workspace. This condensation may provide for or encourage the growth of microorganisms and result in cross-contamination. Because the systems only use cold water, condensation is less likely to form in the surrounding workspace. The systems also reduce the hydraulic load on the waste-water treatment system and eliminate the need to treat the chemicals that would be present in conventional wastewater discharge streams. Ozone creates none of the trihalomethanes commonly associated with chlorine compounds. When properly matched to the application, ozone will reduce most organic compounds to carbon dioxide, water and a little heat. Finally, as ozone sheds the atom of the oxygen causing its molecular instability during the oxidation process, it becomes oxygen again.

### SUMMARY OF THE INVENTION

Ozonated liquid production and distribution system are described. The systems use multiple ozone gas generators to create ozone gas from ambient air. The ozone gas is injected into water or fluid by multiple injectors to form the ozonated liquid.

In one aspect, a variable production and distribution system for ozonated fluid is described. The system includes a water input line and a water input line distributor. The water input line directs water to the water input line distributor. The system includes a first flow sensor line in fluidic communication with the water input line distributor. The first flow sensor line is in fluidic communication with a first flow sensor. The first flow sensor is in fluidic communication with a first injector. The first flow sensor is in electrical communication with a first ozone gas generation unit. The first ozone gas generation unit supplies the first injector with ozone gas. The first injector injects ozone gas into the water to form a first ozonated fluid. The first injector is in fluid communication with a first mixing vessel. The first mixing vessel is in fluidic communication with a main fill line and a first distribution line. The system includes a second flow sensor line in fluidic communication with the water input line distributor. The second flow sensor line is in fluidic communication with a second flow sensor. The second flow sensor is in fluidic communication with a second injector. The second flow sensor is in electrical communication with a second ozone gas generation unit. The second ozone gas generation unit supplies the second injector with ozone gas. The second injector injects ozone gas into the water to form a second ozonated fluid, wherein the second injector is in fluidic communication with a second mixing vessel. The second mixing vessel is in fluidic communication with the main fill line and a second distribution line. The system includes a third flow sensor line in fluidic communication with the water input line distributor. The third flow sensor line is in fluidic communication with a third flow sensor. The third flow sensor is in electrical communication with a third ozone gas generation unit. The third ozone gas generation unit supplies the third injector with ozone gas. The third injector injects ozone gas into the water to form a third ozonated fluid, wherein the third injector is in fluid communication with a third mixing vessel. The third mixing vessel is in fluidic communication with the main fill line and a third distribution line.

In another aspect, a variable production and distribution system for ozonated fluid is described. The system includes a water input line distributor, and the water input line directs water to the water input line distributor. The system includes a plurality of ozonated fluid generators, wherein each ozonated fluid generator comprises a flow sensor line in fluidic communication with the water input line distributor. The flow sensor line is in fluidic communication with a flow sensor. The flow sensor is in fluidic communication with an injector. The flow sensor is in electrical communication with an ozone generation unit. The ozone generation unit supplies the injector with ozone gas. The injector injects the ozone gas into the water to form an ozonated fluid, wherein the injector is in fluid communication with a mixing vessel. Each of the plurality of ozonated fluid generators are in fluid communication with a single fill line and an individual distribution line. The system is operable to direct ozonated fluid to one or more of the individual distribution lines or to the single fill line.

In a further aspect, a system for producing a high volume of ozonated fluid is described. The system includes a water input line and a flow sensor. The water input line is in fluidic communication with the flow sensor. The system includes a water line distributor. The water line distributor separates incoming water from the water input line into a first injector water line and a second injector water line. The system includes a first ozone generation system that includes a first plurality of ozone gas generators and a first ozone supply line. The system includes a second ozone generation system that includes a second plurality of ozone gas generators and a second ozone supply line. The system includes a first injector. A first injector water line is in fluidic communication with the first injector. The first ozone supply line is in supply communication with the first injector. The first injector injects ozone gas into the water to form a first ozonated fluid. The injector supplies a first fill line with the first ozonated fluid. The system includes a second injector. A second injector water line is in fluidic communication with the second injector. The second ozone supply line is in supply communication with the second injector. The second injector injects ozone gas into the water to form a second ozonated fluid. The second injector supplies a second fill line with the second ozonated fluid. The first fill line supplies a main fill line with the first ozonated fluid, and the second fill line supplies the main fill line with the second ozonated fluid.

In a further aspect, a system for producing a high volume of ozonated fluid is described. The system includes a water input line and a flow sensor. The water input line is in fluidic communication with the flow sensor. A first ozonated fluid generator is in fluidic communication with the water input line to supply the generator with water. The first ozonated fluid generator generates a first ozonated fluid. A second ozonated fluid generator is in fluidic communication with the water input line to supply the generator with water. The second ozonated fluid generator generates a second ozonated fluid. The flow sensor is in electrical communication with the first and second ozonated fluid generators. The flow sensor activates the first and second ozonated fluid generators upon sensing flow of water. The first ozonated fluid generator directs to the first ozonated fluid to a fill line. The second ozonated fluid generator directs the second ozonated fluid to the fill line.

### BRIEF DESCRIPTION OF FIGURES

FIG. 1 is a plan view of the variable production and distribution system.

FIG. 2 is a close-up view of the ozone gas generation units.

FIG. 3 is a front view of the mixing vessel.

FIG. 4 is a sectional view of the mixing vessel.

FIG. 5 is a view of the first end of the cone.

FIG. 6 is a perspective view of the spacer.

FIG. 7 is an exploded view of the mixing vessel.

FIG. 8 is a plan view of the high volume ozonated liquid supply system.

FIG. 9 is a graph showing the size distribution of nanobubbles in the ozonated fluid.

FIG. 10 is an image of the light scattering of the nanobubbles in the ozonated fluid.

### DETAILED DESCRIPTION OF THE INVENTION

Systems that produce and distribute an ozonated liquid are described. The systems may be used for cleaning and sanitizing applications and processes. The systems use multiple ozone gas generators to create ozone gas from ambient air. The ozone gas is injected into water or fluid to form the ozonated liquid.

A variable production and distribution system will now be described. The variable product and distribution system produces and distributes an ozonated fluid to one or more distributors, distribution points, such as faucets, sprayers, sinks, etc. or to a single main fill line. The user may determine how the variable distribution system outputs the ozonated fluid. The variable distribution system may simultaneously provide the ozonated fluid to the one or more faucets such that the ozonated fluid may be used simultaneously at one or more work stations, sinks, or cleaning applications. Also, the output of ozonated fluid from the variable distribution system may be directed to the single main fill line such that a large volume of ozonated fluid may be supplied in a relatively short time frame.

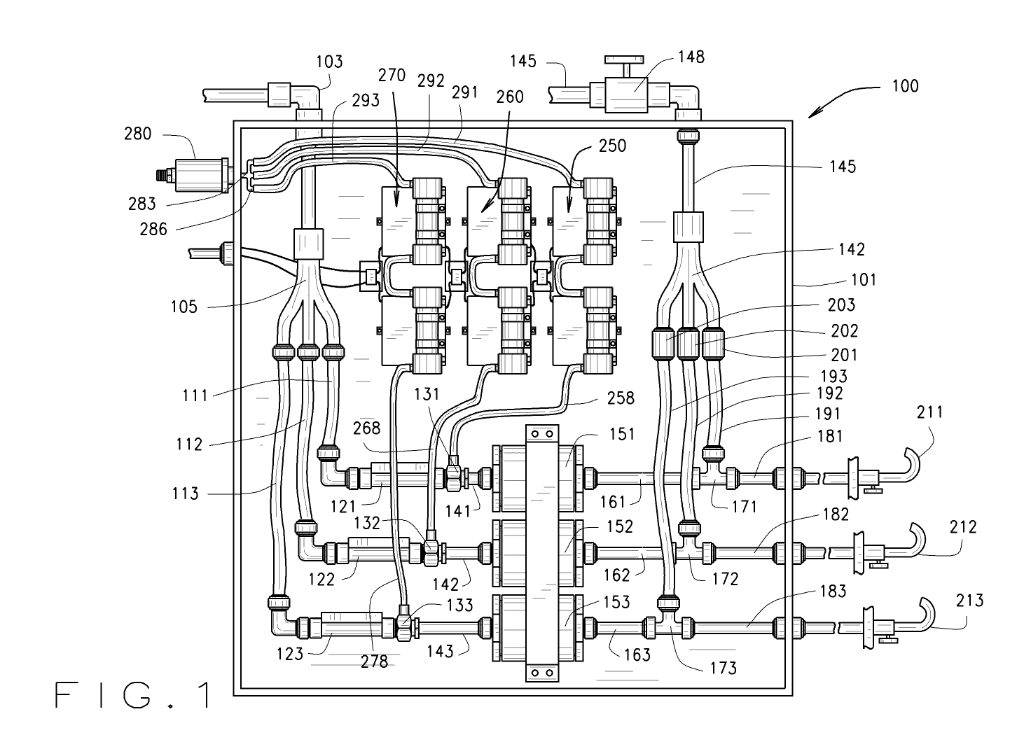

A variable production and distribution system 100 will now be described with reference to FIG. 1. The variable distribution system 100 generates ozone gas, injects the ozone gas into water, and outputs the ozonated fluid in a variable manner that depends on the user's preference. As such, the variable distribution system 100 produces the ozonated fluid on demand and at user-defined flow-rates.

The variable distribution system 100 includes a housing 101. A water input line 103 enters the housing 101 and supplies the variable distribution system 100 with water. The water input line 103 fluidly connects to a water input line distributor 105 that branches into a first flow sensor line 111, a second flow sensor line 112, and a third flow sensor line 113. The flow sensor lines 111, 112, 113 are each in fluidic communication with a first flow sensor 121, a second flow sensor 122, and a third flow sensor 123. The first flow sensor 121 is in electrical communication with a first ozone gas generation unit 250, while the second flow sensor 122 is in electrical communication with a second ozone gas generation unit 260, and the third flow sensor 123 is in electrical communication with a third ozone gas generation unit 270. When water flows through any or all of the flow sensors 121, 122, and 123, the corresponding ozone generation units 250, 260, and 270 are activated to begin producing a supply of ozone gas.

The first flow sensor 121 directs water to a first injector 131. Likewise, the second flow sensor 122 and the third flow sensor 123, respectively, direct the water to a second injector 132 and a third injector 133. The injectors 131, 132, and 133 receive ozone gas produced by the ozone generation units 250, 260, and 270 and inject the ozone gas into the water. Specifically, the first ozone gas generation unit 250 supplies the first injector 131, while the second ozone gas generation unit 260 supplies the second injector 132, and the third ozone gas generation unit 270 supplies the third injector 133 The ozone generation units 250, 260, and 270 are further described below.

The injectors 131, 132, and 133 may be a chemical injector commercially available from Hyrdra Flex, Inc. under the trade name, CHEM FLEX INJECTOR, as part number HF 110057. The injectors 131, 132, and 133 use a check-ball to prevent backflow into the injectors 131, 132, and 133.

From the injectors 131, 132, and 133, the ozonated fluid individually travels to a first mixing vessel input line 141, a second mixing vessel input line 142, and a third mixing vessel input line 143. The mixing vessel input lines 141, 142, and 143 each supply a first mixing vessel 151, a second mixing vessel 152, and a third mixing vessel 153. In the mixing vessels 151, 152, and 153, the ozone fluid is processed to reduce the bubble size of the ozone gas bubbles and to make the bubbles have a uniform size.

From the mixing vessels 151, 152, and 153, the ozonated fluid passes to a first mixing vessel output line 161, a second mixing vessel output line 162, and a third mixing vessel output line 163. Each of the mixing vessel output lines 161, 162, and 163 include a splitting connection 171, 172, and 173. The splitting connections 171, 172, and 173 split the flow from the respective mixing vessel 151, 152, and 153 to either the main fill line 145, via fill supply lines 191, 192, 193, or to one or more distribution lines 181, 182, 183.

With respect to the first mixing vessel 151, the ozonated fluid passes through the first mixing vessel output line 161. The first splitting connection 171 directs the ozonated fluid to the first fill supply line 191 and to the first distribution line 181.

The first fill supply line 191 includes a first backflow preventer 201. The backflow preventer 201 assists the system 100 in maintaining pressure such that the fluid will pass to the first distribution line 181 and on to a first distributor 211. The backflow preventer 201 allows the proper pressure to be maintained when the first distributor 211 is opened. For example, when a user opens the first distributor 211, fluid begins to flow through the first mixing vessel output line 161 and is directed either to the first distributor 211 or the first fill supply line 191. Without the first backflow preventers 201, 202, and 203 positioned in the supply lines 191, 192, and 193, the pressure from the water source would have to pass all the way through the entire second distribution line 182 and the third distribution line 183 in order to cause the fluid to dispense from the first distributor 211. Each of the distribution lines 181, 182, and 183 could be many feet in length. By providing the backflow preventers 201, 202, and 203, the system 100 only has to pressurize the relatively short length between the backflow preventers 201, 202, and 203 and the splitting connections 171, 172, and 173.

In a closed position, i.e., when the main fill line 145 and the distributors 211, 212, and 213 are all closed, the incoming water supply through the water input line 103 charges the entire system 100 with pressure such that the opening of any of the distributors 211, 212, or 213 or the main fill line valve 148, will cause ozonated fluid to flow from the respective opened line.

When the main fill line valve 148 is opened, all three of the ozone gas generation units 250, 260, and 270 begin producing ozone gas that is supplied to the respective injector 131, 132, or 133. With the distributors 211, 212, or 213 all in the closed position, all of the output of the ozonated fluid is directed to the main fill line 145. The main fill line reducer 142 joins the fluid flow from each of the fill supply lines 191, 192, and 193.

The ozone gas generation units 250, 260, and 270 may draw air from the surrounding environment or be supplied with oxygen gas from an oxygen concentrator or generator in order to generate ozone gas. The ozone gas generation unit 250 includes a first dielectric cell 251 and a second dielectric cell 252. Similarly, the ozone gas generation unit 260 includes a first dielectric cell 261 and a second dielectric cell 262, and the ozone gas generation unit 270 includes a first dielectric cell 271 and a second dielectric cell 272.

The housing 101 may further include an air dryer 280. Air used by the system 100 may first pass through an air dryer 280 to remove moisture from the air to increase the efficiency of the ozone gas generation units 250, 260, and 270. A suitable dryer 280 is commercially available from SPEEDAIR as model 6ZC63.

From the air dryer 280, the air is passed to a main air line 283. The main air line 283 includes an air line distributor 286 that branches the main air line 283 into a first air line 291, a second air line 292, and a third air line 293. The first air line 291 supplies the first ozone gas generation unit 250, while the second air line 292 supplies the second ozone gas generation unit 260, and the third air line 293 supplies the third ozone gas generation unit 270.

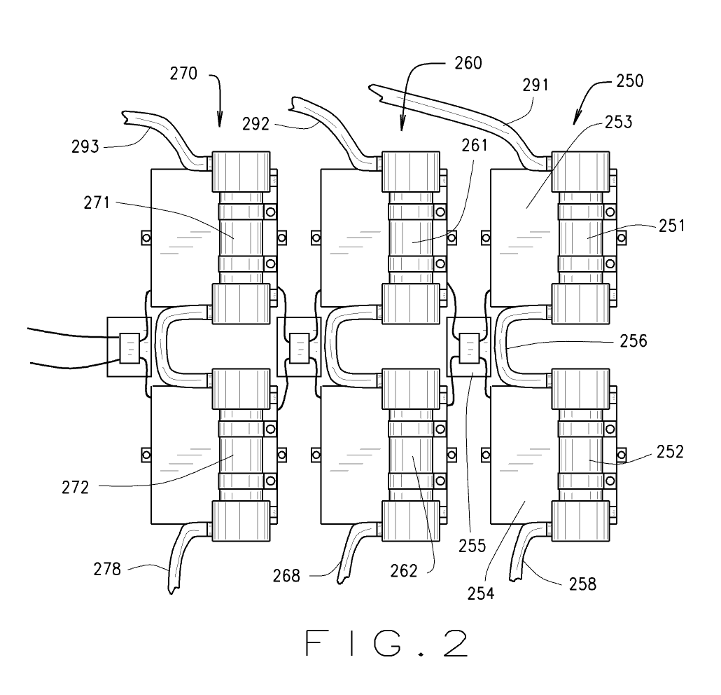

With respect to FIG. 2, the first air line 291 supplies air to the first dielectric cell 251. The first dielectric cell 251 makes ozone gas from the air passing through the first dielectric cell 251. The first dielectric cell 251 includes a glass or other insulating cylinder. An electrical conductor passes through the cylinder. A conductive metal lattice, metal mesh, or coil wire surrounds the conductor. When power is supplied to the first dielectric cell 251, electricity passes through the conductor and sparks and arcs. This electrical discharge splits the oxygen molecules creating ozone gas from oxygen molecules present in the supply gas inside of the first dielectric cell 251. This method is generally referred to as corona discharge.

The ozone gas from the first dielectric cell 251 is passed to the second dielectric cell 252 as a supply gas. The second dielectric cell 252 is constructed similar to the first dielectric cell 251. At the second dielectric cell 252, additional ozone gas is generated from the supply gas.

The first and second dielectric cells 251 and 252 are commercially available from Alta Industries as models PA-021. Each of the first and second dielectric cells 251 and 252 may provide 250 mg of ozone gas per hour. Power cells 253 and 254 supply the first and second dielectric cells 251 and 252 with the power. A relay 255 electrically connects the power cells 253 and 254 with the first flow sensor 121. The relay 255 may include a button pack relay commercially available from Compaq Engineering Inc.

As described above, ozone gas created by the coronal discharge in the first dielectric cell 251 is captured and supplied to the second dielectric cell 252. The supply gas from the first dielectric cell 251 to the second dielectric cell 252 contains an amount of ozone gas. In detail, the first air line 291 connects to a first gas input trap of the first dielectric cell 251. An output end of the first dielectric cell 251 is sealingly connected to and surrounded by a first gas output trap. The first gas output trap funnels the ozone gas created by the first dielectric cell 251 to a gas line 256 which is in fluidic communication with a second gas input trap of the second dielectric cell 252. The gas line 256 thus connects to the first gas output trap and to the second gas input trap. The second gas input trap is sealingly connected to a first or input end of the second dielectric cell 252. As such, supply gas to the second dielectric cell 252 already includes a first amount of ozone gas. The supply gas from the first dielectric cell 251 is further processed by the second dielectric cell 252 to add an additional amount of ozone gas to the supply gas.

The first gas output trap seals the output of ozone gas from the first dielectric cell 251 such that nearly all of the ozone gas created by the first dielectric cell 251 or the output of gas from the first dielectric cell 251 is supplied in a closed communication via gas line 256 to the second dielectric cell 252. The closed communication provides for the second dielectric cell 252 to form ozone gas from the output gas of the first dielectric cell 251.

The second gas output trap is sealingly connected to a second or output end of the second dielectric cell 252. The ozonated gas produced by the second dielectric cell 252 is transported via an injector supply line 258 to the injector 131.

When the first distribution line 181 is opened, water enters the system 100 at the water input line 103. The water is directed through the first flow sensor 121, which activates the first dielectric cell 251 and the second dielectric cell 252. The water may simultaneously activate the first dielectric cell 251 and the second dielectric cell 252. A suitable flow switch for the first flow sensor 121 includes a straight body polypropylene flow switch, such as a Series 5 ERECTA switch commercially available from OKI Sensor Device Corporation. Next, the water passes to the injector 131, which injects the water with the ozone gas.

The first flow sensor 121 is positioned in the water flow before the water reaches the injector 131. From the injector 131, the ozonated fluid passes through a connector 141, which passes the ozonated fluid to the first mixing vessel 151, for additional mixing and processing. The further processed and mixed ozonated fluid exits the first mixing vessel 151.

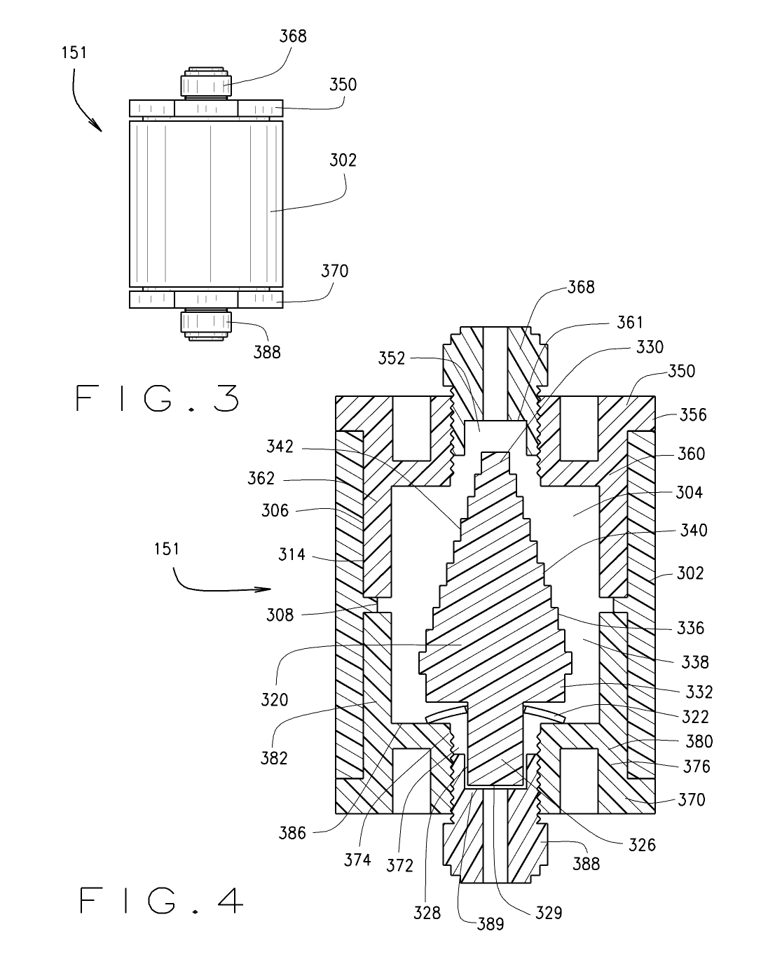

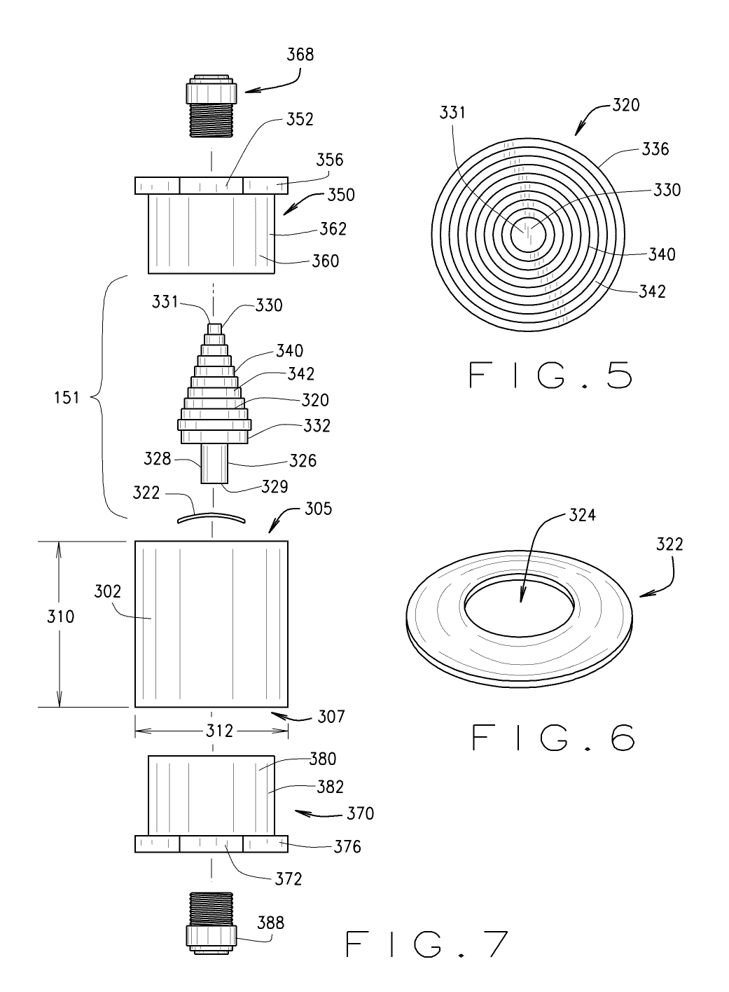

The first mixing vessel 151 will now be described with reference to FIGS. 3-7, in which the first mixing vessel 151 is shown in detail. The second and third mixing vessels 152 and 153 may be constructed similarly.

The mixing vessel 151 generally includes a housing 302, a cone 320, a first end 350, and a second end 370. The ozonated solution enters the housing 302 via the first end 350. The housing 302 has a generally hollow interior 304. The cone 320 is positioned in the interior 304 of the housing 302. The ozonated solution flows past and against the cone 320 and exits the housing 302 via the second end 370. During assembly of the mixing vessel 151, the first end 350 is inserted into a first housing opening 305 of the housing 302, while a second end 370 is inserted into a second housing opening 307 of the housing 302. The cone 320 is positioned in the interior 304 of the housing 302 and is loosely held in position by the first end 350 and the second end 370.

The housing 302 has an inner surface 306 that is generally smooth except for an annular ridge 308, which extends into the interior 304 of the housing 302. As shown in an exploded view of the mixing vessel 151 in FIG. 5, an insert portion 360 of the first end 350 is inserted into the first housing opening 305 of the housing 302 until the insert portion 360 approximately abuts the annular ridge 308. Likewise, an insert portion 380 of the second end 370 is inserted into the second housing opening 307 until the insert portion 380 approximately abuts an opposite surface of the annular ridge 308. As such, the insert portions 360 and 380 fit into opposite ends of the housing 302, and the cone 320 is positioned between the opposite ends.

The housing 302 has a generally cylindrical shape having a length 310 that is generally greater than a width 312. The housing 302 has an inner diameter 314 that is greater than an outer diameter 336 of the cone 320. The first end 350 generally includes a rim portion 356 and the insert portion 360. Likewise, the second end 370 generally includes a rim portion 376 and the insert portion 380.

The insert portions 360 and 380 of the first end 350 and the second end 370 include walls 362 and 382, respectively, which are positioned flush against the interior surface 306 of the housing 302. A frictional engagement may hold the walls 362 and 382 against the interior surfaces 306 of the housing 302. Glue, adhesives, epoxy, sealants, etc. may also be used to seal the housing 302 with the walls 362 and 382.

The first end 350 also includes a central opening 352 that allows the ozonated fluid to enter the housing 302. The second end 370, likewise, has a central opening 372 that allows the ozonated fluid to exit from the housing 302. A first coupling 368 and a second coupling 388 may fit into the central openings 352 and 372, respectively, in order to plumb or fluidly connect the mixing vessel 151 with the remainder of the system or apparatus that is producing the ozonated fluid.

The cone 320 will now be described with reference to FIG. 16. The cone 320 includes a first end 330 opposite of a second end 332. The cone 320 is positioned between the central openings 352 and 372. The first end 330 generally has a smaller external diameter than an external diameter of the second end 332.

The cone 320 further includes a stem 326. The stem 326 extends from the second end 332 of the cone 320. The stem 326 may be integral with the second end 332 of the cone 320. An exterior of the cone 320 includes a contact surface 340. The contact surface 340 may include a plurality of ridges or steps 342 that contact the ozonated fluid. The plurality of ridges or steps 342 may progressively increase in diameter from the first end 330 to the second end 332. The plurality of ridges or steps 342 of the contact surface 340 assist in breaking up the bubbles of ozone gas in the ozonated fluid and further mixing the ozone gas with the water. The contact surface 340 may include 3 to 30 or more ridges or steps 342. The number of ridges or steps 342 may depend on the size of the cone 320 and particular application in which the cone 320 is used.

The cone 320 generally increases in external diameter from the first end 330 to the second end 332. During use of the mixing vessel 151, the ozonated fluid is directed into the mixing vessel 151 such that the first end 330 of the cone 320 is positioned in the incoming flow of the ozonated fluid. As such, the ozonated fluid is first directed against the smaller first end 330 of the cone 320. The ozonated fluid flows against the contact surface 340 of the cone 320. The ozonated fluid circulates about the contact surface 340 to break up the bubbles of ozone gas against the contact surface 340. The cone 320 breaks up the bubbles to create unformed size bubbles. The ozonated fluid passes the cone 320 in an annular fluid passage 338 that is formed between the contact surface 340 of the cone 320 and the combination of the inner surface 306 of the housing 302 and the walls 362 and 382 of the first insert portion 360 and the second insert portion 380, respectively.

The stem 326 of the cone 320 loosely fits into the central opening 372 of the second end 370. A spacer 322 is fitted over the stem 326. The spacer 322 has a spacer opening 324 that receives the stem 326. The spacer opening 324 is slightly larger than an outer diameter 328 of the stem 326. The spacer 322 has a generally curved or angled shape to position the second end 332 of the cone 320 spaced away from the central opening 372 such that the second end 332 of the cone 320 does not block or fully occlude the central opening 372, which allows the ozonated fluid to pass through the central opening 372. The spacer 322 may rest against an end surface 386 of the insert portion 380. The outer diameter 328 of the stem 326 is smaller than a central opening wall 374 of the central opening 372 to provide a fluid passage between the outer diameter 328 of the stem 326 and the central opening wall 374.

The cone 320 has an overall length that provides for the first end 330 of the cone 320 to enter the central opening 352 while the stem 326 enters the central opening 372. This arrangement loosely holds the stem 326 in its intended position, i.e., the cone 320 is held generally centrally in the housing 302 and general vertically aligned with respect to the length 310 of the housing 302. The cone 320 generally has a solid shape. The cone 320 may be molded from a variety of conventional thermoplastics.

The first coupling 368 and the second coupling 388 may be inserted or threadably attached to the central openings 352 and 372, respectively. Each of the first coupling 368 and the second coupling 388 may include a recessed portion 369 and 389, respectively that receives the tip 331 of the first end 330 or a base surface 329 of the stem 326. The first coupling 368 may fluidly connect to hoses, tubing, conduits, passages, pipes, etc. that supply the mixing vessel 151 with the ozonated fluid. The second coupling 388 may fluidly connect to hoses, tubing, conduits, passages, pipes, etc. to output the processed ozonated fluid from the mixing vessel 151. Each of the first coupling 368 and the second coupling 388 include a fluid passage or opening.

The system 100 provides a flow rate of approximately ½ gallon per minute at a concentration of approximately 2.0 ppm to approximately 3.0 ppm. The system 100 may be scaled up or down to increase or decrease the amount of flow of ozonated fluid. The system 100 may be integrated or incorporated into a variety of systems or platforms that spray or apply an ozonated fluid in order to clean, sanitize, disinfect, etc.

A high volume ozonated liquid supply system will now be described. The high volume supply system uses multiple ozone gas generation units and multiple injectors to supply a high volume of ozonated fluid on demand. Multiple ozone generation units are supplying each of the multiple injectors in order to create the supply of ozone gas needed. Multiple supplies of ozonated fluid are produced on demand and combined to provide the high volume of ozonated fluid. The high volume supply system is useful for supplying high volumes of ozonated liquid on demand. For example, the high volume supply system may be used to fill a bucket, holding tank, or other wash receptacle full of ozonated fluid. The high volume supply system may be used to quickly fill a soaking tank or the like with ozonated fluid.

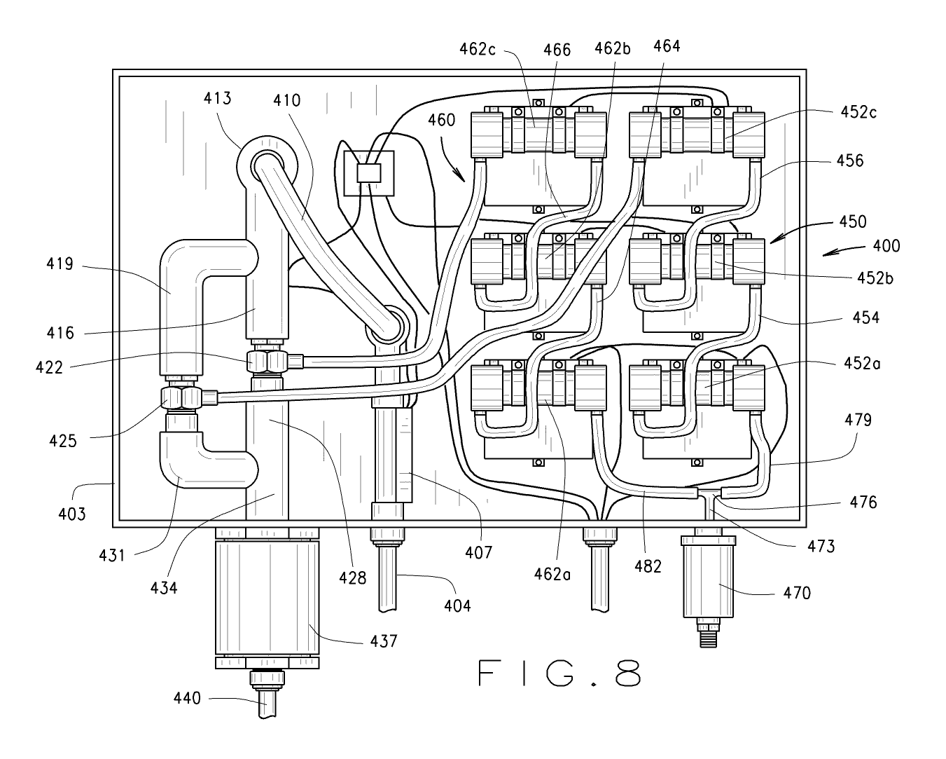

The high volume supply system will now be described with reference to the figures. A high volume supply system 400 is shown in FIG. 8. A water input line 404 supplies fresh water to the high volume supply system 400. Upon entering the supply system 400, the water from the water input line 404 passes through a flow sensor 407, which triggers the generation of ozone gas by a first ozone gas generation system 450 and a second ozone generation gas system 460.

From the flow sensor 407, the water passes through a water line 410 to a water line distributor 413. The water line distributor 413 separates the incoming water from the water line 410 to a first injector line 416 and a second injector line 419. The first injector line 416 includes a first injector 422. Likewise, the second injector line 419 includes a second injector 425. The first injector 422 is in fluidic communication with the first ozone gas generation system 450 that provides ozone gas to the first injector 422. The second injector 425 is in fluidic communication with the second ozone gas generation system 460 that provides ozone gas to the second injector 425.

The first injector 422 leads to a first fill line 428, and likewise, the second injector 425 leads to a second fill line 431. The first fill line 428 and the second fill line 431 join at a fill line reducer 434. The ozonated fluids formed at the first injector 422 and at the second injector 425 are combined into one fluid at the fill line reducer 434. The fill line reducer 434 is in fluidic communication with a mixing vessel 437.

The mixing vessel 437 mixes and processes the ozonated gas and the ozonated fluids to provide a consistent and uniform bubble size for the ozone gas. The mixing vessel 437 may be formed similar to the mixing vessel 151, 152, and 153 herein described.

From the mixing vessel 437, the ozonated fluid passes to a main fill line 440. The main file line 440 may include a nozzle, spigot, hosing, etc. The main fill line 440 may include a valve or other regulating structure. When the valve is opened, the production of ozonated fluid in the high volume supply system 400 begins. The high volume supply system 400 may provide a supply of ozonated fluid at approximately ½ to approximately 10 gallons per minute at an oxidation reduction potential of approximately 700 millivolts to approximately 1000 millivolts.

The housing 403 further includes an air dryer 470. Air passing into the high volume supply system 400 first passes through an air dryer 470 to remove moisture from the air to increase the efficiency of the first and second ozone gas generation systems 450 and 460. The main air line 473 includes an air line distributor 476 that branches the main air line 473 into the first air line 479 and the second air line 482. The first line 479 supplies the first ozone gas generation system 450, while the second line 482 supplies the second ozone gas generation system 460.

The first ozone gas generation system 450 includes three separate ozone generators all plumbed in a serial configuration. Likewise, the second ozone gas generation system 460 includes three separate ozone generators all plumbed in a serial configuration. As shown in FIG. 8, the first ozone gas generation system 450 includes a first ozone gas generator 452A, a second ozone gas generator 452B, and a third ozone gas generator 452C. The ozone gas generators 452A, 452B, and 452C are plumbed in a serial configuration such that the first line 479 supplies the first ozone gas generator 452A, and the output of the first ozone gas generator 452A supplies the second ozone gas generator 452B via a second line 454. Finally, the output of the second ozone gas generator 452B supplies the third ozone gas generator 452C via a third line 456. Similarly, the second ozone gas generation system 460 includes a first ozone gas generator 462A, a second ozone gas generator 462B, and a third ozone gas generator 462C. The ozone gas generators 462A, 462B, and 452C are also plumbed in a serial configuration such that a first line 482 supplies the first ozone gas generator 462A, and the output of the first ozone gas generator 462A supplies the second ozone gas generator 462B via a second line 464. Finally, the output of the second ozone gas generator 462B supplies the third ozone gas generator 462C via third line 466.

During production of the high volume of ozonated fluid, both of the ozone gas generation systems 450 and 460 are simultaneously generating ozone gas and supplying the ozone gas to their respective injectors 422 and 425. The injectors 422 and 425 are also simultaneously injecting ozone gas into two different streams of water.

The flow sensor 407 triggers the generation of ozone gas by both of the first ozone gas generation system 450 and a second ozone generation gas system 460. The flow sensor 407 is in electrical communication with the first ozone gas generation system 450 and the second ozone generation gas system 460 to simultaneously activate the first ozone gas generation system 450 and the second ozone generation gas system 460 upon sensing fluid flow. As such, the single flow sensor 407 initiates production of ozonated fluid at the injectors 422 and 425 and two supplies of ozonated fluid are provided to the first fill line 428 and the second fill line 431.

Although the high volume supply system 400 includes the two ozone gas generation systems 450 and 460 each having three ozone generators, the number of ozone gas generation systems employed in the high volume supply system 400 may be increased and the number of individual ozone gas generators per ozone generation system may also be increased.

Ozonated fluids produced by the variable production and distribution system 100 were analyzed. During the production of the ozonated fluid, the mixing vessels 151, 152 and 153 reduce a bubble size of ozone gas bubbles to create uniform-sized nanobubbles with a spherical geometry and thereby lowering the surface tension in the ozonated fluid. The nanobubbles may have a diameter of less than 300 nanometers. Cohesive forces among liquid molecules are responsible for the phenomenon of surface tension in the liquid. In the bulk of the liquid, each molecule of liquid is pulled equally in every direction by neighboring liquid molecules, resulting in a net force of zero. The molecules at the surface of the liquid do not have other molecules on all sides of them and therefore are pulled inwards. This creates some internal pressure and forces liquid surfaces to contract to the minimal area. As a result of this surface area minimization, a surface will assume the smoothest shape it can. Mathematical proof that “smooth” shapes minimize surface area relies on use of the Euler-Lagrange equation. Since any curvature in the surface shape results in greater area, a higher energy will also result, which in turn produces a higher oxidation-reduction potential for the ozonated fluid.

As the amount of oxidizer in the water is increased, the oxidizer “steals” electrons from the surface of a platinum measuring electrode, which is used to measure oxidation-reduction potential. When these negatively charged electrons are removed from this electrode, the electrode becomes more and more positively charged. As more oxidizer is added to the water, the electrode generates a higher and higher positive voltage. Consequently, the surface will push back against any curvature to minimize its gravitational potential energy. Surface tension is visible in other common phenomena, especially when surfactants are used to decrease it.

Ozone gas bubbles have very large surface areas with very little mass. Bubbles in pure water are unstable. Lowering the surface tension results in having a stabilizing effect on the bubbles, which is described by the Marangoni effect. This process makes the ozononated fluid acts like surfactant and actually reduces the surface tension by a factor of three or more. This process makes the ozonated fluid a type of emulsion and causes the solution to have a degreasing as well as a sanitizer effect on applied surfaces. Surface tension in the ozonated fluid water creates a sheet of ozonated fluid between the flow and the surface of the ozonated fluid. The surface of the ozonated fluid behaves like an elastic film increasing its surface area. The surface tension of water at 20 C is 72.8 mN/m. The surface tension results of the tested ozonated fluid, branded VIRIDITEC, was 49.1 mN/m or equal to the surface tension of hot water 60 C. The table below shows how the internal pressure of a water droplet increases with decreasing radius. For not very small drops the effect is subtle, but the pressure difference becomes enormous when the drop sizes approach the molecular size. (In the limit of a single molecule, the concept becomes meaningless.)

| Droplet radius | 1 mm | 0.1 mm | 1 μm | 10 nm |
|---|---|---|---|---|
| Δp (atm) | 0.0014 | 0.0144 | 1.436 | 143.6 |

In the above tests, a Sigma Tensionmeter 701 was used to measure the surface tension. The Sigma tensionmeter 701 uses a Wilhelmy plate consisting of a thin plate usually on the order of a few square centimeters in area. The plate is often made from glass or platinum which may be roughened to ensure complete wetting. The plate is cleaned thoroughly and attached to a scale or balance via a thin metal wire. The force on the plate due to wetting is measured via a tensionmeter or microbalance and used to calculate the surface tension using the Wilhelmy equation:

$$γ = \frac{F}{l \cdot cos θ}$$

where l is the wetted perimeter (2w+2d) of the Wilhelmy plate and θ is the contact angle between the liquid phase and the plate. In practice the contact angle is rarely measured, instead either literature values are used, or complete wetting (θ=0) is assumed. When calculating surface tensions when using the Wilhelmy plate, a zero contact angle is assumed. In addition, because the plate is not moved during measurements, the Wilhelmy plate allows accurate determination of surface kinetics on a wide range of timescales and it displays low operator variance. In a typical plate experiment, the plate is lowered to the surface being analyzed until a meniscus is formed, and then raised so that the bottom edge of the plate lies on the plane of the undisturbed surface. If measuring a buried interface, the second (less dense) phase is then added on top of the undisturbed primary (denser) phase in such a way as to not disturb the meniscus. The force at equilibrium can then be used to determine the absolute surface or interfacial tension. Surface tension is therefore measured in forces per unit length. Its SI unit is newton per meter but the CGS unit of dyne per cm is also used. One dyn/cm corresponds to 0.001 N/m.

The ozonated fluids produced by the variable production and distribution systems 100 were also analyzed to measure zeta potential. The significance of zeta potential is that its value can be related to the stability of the ozonated solution. The zeta potential indicates the degree of repulsion between adjacent, similarly charged particles in a solution. For molecules and particles that are small enough, a high zeta potential will confer stability, i.e., the solution or dispersion will resist aggregation. When the potential is low, attraction exceeds repulsion and the dispersion will break and flocculate. So, colloids with high zeta potential (negative or positive) are electrically stabilized while colloids with low zeta potentials tend to coagulate or flocculate as outlined in the table.

Zeta potential [mV] Stability behavior of the colloid

| | |
|---|---|
| from 0 to ±5, | Rapid coagulation or flocculation |
| from ±10 to ±30 | Incipient instability |
| from ±30 to ±40 | Moderate stability |
| from ±40 to ±60 | Good stability |
| more than ±61 | Excellent stability |

Although the ozonated fluid had a high Zeta potential of 60 indicating excellent stability for the ozonated fluid, limitations of the dynamic light scattering (DLS) tests on the ozonated fluid did not allow for good results in measuring the bubble size in nanometers. It is concluded that the low surface tension of the ozonated fluid clouds the DLS size measurement because the surface tension creates a sheet of ozonated fluid between the flow and the surface. The surface of the ozonated fluid behaves like an elastic film resulting in blocking the bubble size function of the DLS test. It is concluded that the bubble size is in the 100 to 500 nm range because of the high Zeta Potential.

A sample of ozonated fluid produced by the variable production and distribution system 100 was further analyzed using a Nanosight LM10-HSGT nano-particle visualization device, which employs the Nanoparticle Tracking Analysis technique as defined in ASTM 2834-12. With this device, individual particles, i.e., the ozone nanobubbles in the ozonated fluid, are visualized down to approximately 10 nanometer in diameter. The device is not imaging the ozone nanobubbles at this size scale, i.e., no structural or shape information is available. Instead, the ozone nanobubbles are being visualized through the light that the particles scatter.

The concentration of particles for the sample was within the measurement range of the device and no dilution or other sample treatment was required. In this case, the “particles” are the nanobubbles of ozone, which scatter light and undergo Brownian motion in the same manner as a solid particle.

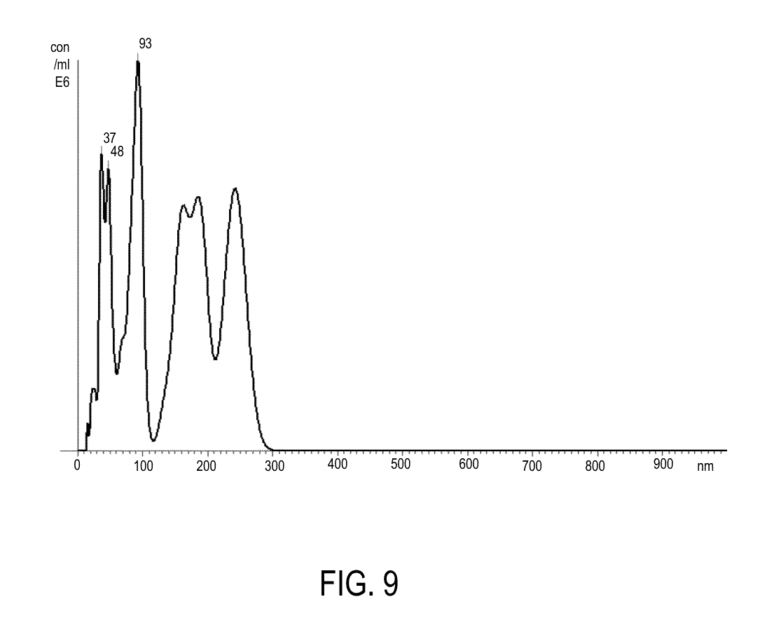

The sample of ozonated fluid showed a range of the ozone nanobubbles with a lower size limit around approximately 30 nm and a tail of the larger sizes up to approximately 300 nm. A graph showing the results is shown in FIG. 9. The results are also shown in the table below:

| Mode Size | | | | Concentration |
|---|---|---|---|---|
| (nm) | D10 | D50 | D90 | (×10\hat{ }8 particles/ml) |
| 93 | 46 | 162 | 248 | 0.38 |

The analysis of the ozonated fluid showed that 10% of the ozone nanobubbles had a diameter less than 46 nanometers, 50% of the ozone nanobubbles had a diameter less than 162 nanometers, and 90% of the ozone nanobubbles had a diameter less than 248 nanometers. The analysis showed a mode size of 93 nanometers. No ozone nanobubbles having a diameter greater than 300 nanometers were found present in the ozonated fluid. As such, the ozonated fluid from the variable production and distribution system 100 contained ozone nanobubbles having a diameter of approximately 30 nanometers to approximately 300 nanometers. The ozonated fluid from the system 100 contained only ozone nanobubbles having a diameter less than approximately 300 nanometers.

During the analysis, 300 μl of the ozonated fluid from the variable production and distribution system 100 was introduced into the sample cell of the NanoSight LM10-HSGT using a 1 ml disposable syringe and visualized using a conventional optical microscope (×20 long working distance objective 0.40 NA) fitted with a scientific video camera (Hamamatsu CMOS). Images were collected directly to the hard drive as *.avi files with no further image processing.

The NanoSight LM10-HSGT uses a 532 nm, 50 mW laser to pass a laser beam through a prism-edged optical flat, the refractive index of which is such that the beam refracts at the interface between the flat and a liquid layer of the ozonated fluid placed above it. Due to the refraction, the beam compresses to a low profile, intense illumination region in which nanoparticles present in the liquid film can be easily visualized via the microscope. Mounted on a C mount, the CMOS camera, operating at 30 frames per second, is used to capture a video field of view approximately 100 μm×80 μm.

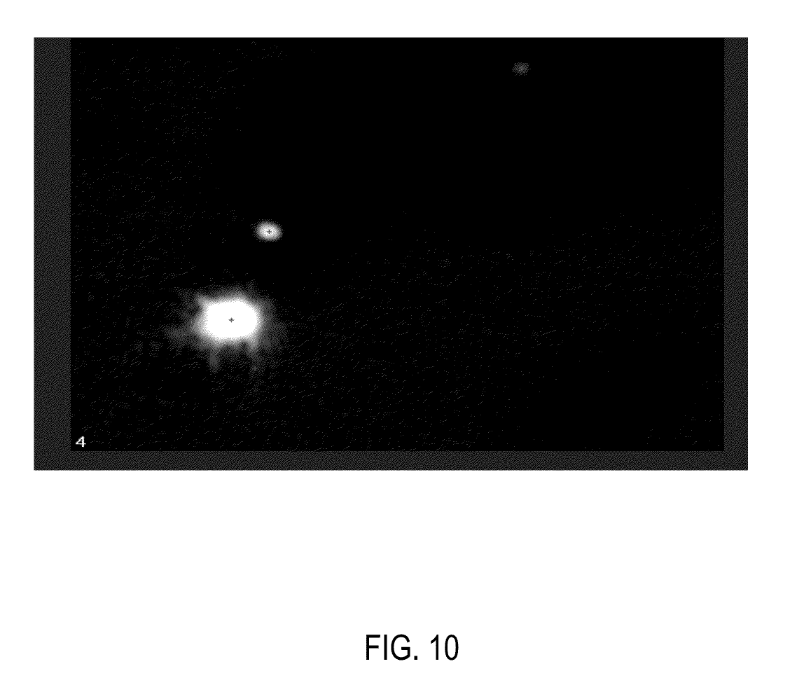

Particles in the scattering volume are seen moving rapidly under Brownian motion. A software program simultaneously identifies and tracks the center of each particle on a frame-by-frame basis throughout the length of the video. A sample image from the video is shown as FIG. 10. The image is not a direct image of the ozone nanobubbles themselves, instead the image shows the light scattered by the ozone nanobubbles.

The average distance each particle moves in x and y in the image is automatically calculated, from which the diffusion coefficient (Dt) and hence sphere-equivalent, hydrodynamic diameter (d) can be determined using the Stokes-Einstein equation:

$$Dt = \frac{{K}_{B} T}{3 πη d}$$

where K_{B} is Boltzmann's constant, T is temperature and η is viscosity.

The scattering intensity of a particle is dependent upon its size (with larger particles scattering more light) and also its refractive index. The Brownian motion however, is dependent only upon the particle size, solvent viscosity and temperature (and is absolutely independent of particle density) and therefore provides an absolute measure of particle size, with smaller particles having a more exaggerated motion.

The intensity or brightness of the particles in the image may not necessarily indicate the presence of larger particles since the intensity of the particles may be associated with the refractive index of the particle. The video image, however, may be used to qualitatively asses the size of the particle both by the intensity and its Brownian motion (with the Brownian motion only being used to size the particle).

Those skilled in the art will appreciate that variations from the specific embodiments disclosed above are contemplated by the invention. The invention should not be restricted to the above embodiments, but should be measured by the following claims.

What is claimed is:

1. A variable production and distribution system for ozonated fluid, comprising:
a water input line;
a water input line distributor, and the water input line directs water to the water input line distributor;
a first flow sensor line in fluidic communication with the water input line distributor, the first flow sensor line in fluidic communication with a first flow sensor; the first flow sensor in fluidic communication with a first injector; the first flow sensor in electrical communication with a first ozone gas generation unit; the first ozone gas generation unit supplies the first injector with ozone gas; the first injector injects ozone gas into the water to form a first ozonated fluid, wherein the first injector is in fluid communication with a first mixing vessel, the first mixing vessel is in fluidic communication with a main fill line and a first distribution line;
a second flow sensor line in fluidic communication with the water input line distributor, the second flow sensor line in fluidic communication with a second flow sensor; the second flow sensor in fluidic communication with a second injector; the second flow sensor in electrical communication with a second ozone gas generation unit; the second ozone gas generation unit supplies the second injector with ozone gas; the second injector injects ozone gas into the water to form a second ozonated fluid, wherein the second injector is in fluidic communication with a second mixing vessel, the second mixing vessel is in fluidic communication with the main fill line and a second distribution line; and,
a third flow sensor line in fluidic communication with the water input line distributor, the third flow sensor line in fluidic communication with a third flow sensor; the third flow sensor in electrical communication with a third ozone gas generation unit; the third ozone gas generation unit supplies the third injector with ozone gas; the third injector injects ozone gas into the water to form a third ozonated fluid, wherein the third injector is in fluid communication with a third mixing vessel, the third mixing vessel is in fluidic communication with the main fill line and a third distribution line.

2. The variable production and distribution system according to claim 1, wherein the system outputs the ozonated fluid in a variable manner that depends on a user's preference.

3. The variable production and distribution system according to claim 1, wherein the system outputs the first, second, and third ozonated fluids from the main fill line.

4. The variable production and distribution system according to claim 1, wherein the first mixing vessel passes the first ozonated fluid to a first mixing vessel output line, the second mixing vessel passes the second ozonated fluid to a second mixing vessel output line, and the third mixing vessel passes the third ozonated fluid to a third mixing vessel output line, and each of the mixing vessel output lines include a splitting connection, and the splitting connections split the flow from the mixing vessels to either the main fill line or to one of the distribution lines.

5. The variable production and distribution system according to claim 1, wherein the first mixing vessel passes the first ozonated fluid to a first mixing vessel output line, the second mixing vessel passes the second ozonated fluid to a second mixing vessel output line, and the third mixing vessel passes the ozonated fluid to a third mixing vessel output line, and wherein the first mixing vessel output line includes a first splitting connection, and the first splitting connection splits the flow from the first mixing vessel to either the main fill line or to the first distribution line, wherein the second mixing vessel output line includes a second splitting connection, and the second splitting connection splits the flow from the second mixing vessel to either the main fill line or to the second distribution line, and wherein the third mixing vessel output line includes a third splitting connection, and the third splitting connection splits the flow from the third mixing vessel to either the main fill line or to the third distribution line.

6. The variable production and distribution system according to claim 1, wherein a first fill supply line fluidly connects the first mixing vessel with the main fill line, and the first fill supply line includes a first backflow preventer; wherein a second fill supply line fluidly connects the second mixing vessel with the main fill line, and the second fill supply line includes a second backflow preventer, wherein a third fill supply line fluidly connects the third mixing vessel with the main fill line, and the third fill supply line includes a third backflow preventer.

7. The variable production and distribution system according to claim 1, wherein the first fill supply line, the second fill supply line, and the third fill supply line all fluidly connect with a main fill line reducer to join the fluid flow from each of the fill supply lines.

8. The variable production and distribution system according to claim 1, wherein the first fill supply line, the second fill supply line, and the third fill supply line all fluidly connect with a main fill line reducer, the main fill line reducer is in fluidic communication with the main fill line, and wherein first fill supply line supplies the main fill line with the first ozonated fluid, wherein the second fill supply line supplies the main fill line with the second ozonated fluid, and wherein the third fill supply line supplies the main fill line with the third ozonated fluid.

9. The variable production and distribution system according to claim 1, wherein the system is fluidly connected to a water supply to supply the system with water, wherein water pressure from the water supply pressurizes the system.

10. The variable production and distribution system according to claim 1, wherein the main fill line and each of the distribution lines have an open and a closed position, wherein a water supply, through the water input line, charges the system with pressure such that the opening of any of the distribution lines or the main fill line will cause ozonated fluid to flow from the respective opened line.

11. The variable production and distribution system according to claim 1, wherein the main fill line and each of the distribution lines have an open and a closed position, wherein a water supply, through the water input line, charges the system with pressure such that the opening of any of the distribution lines or the main fill line will cause ozonated fluid to flow from the respective opened line, and wherein opening the main fill line causes all of the first ozone gas generation unit, the second ozone gas generation unit, and the third ozone gas generation unit to begin producing ozone gas.

12. The variable production and distribution system according to claim 1, wherein the main fill line and each of the distribution lines have an open and a closed position, wherein a water supply, through the water input line, charges the system with pressure such that the opening of any of the distribution lines or the main fill line will cause ozonated fluid to flow from the respective opened line, and wherein closing all of the distribution lines will direct all ozonated fluid to the main fill line.

13. The variable production and distribution system according to claim 1, wherein the first distribution line, the second distribution line, and the third distribution line simultaneously provide an ozonated fluid.

14. A variable production and distribution system for ozonated fluid, comprising:
a water input line distributor, and the water input line directs water to the water input line distributor;
a plurality of ozonated fluid generators, wherein each ozonated fluid generator comprises a flow sensor line in fluidic communication with the water input line distributor, the flow sensor line in fluidic communication with a flow sensor; the flow sensor in fluidic communication with an injector; the flow sensor in electrical communication with an ozone generation unit; the ozone generation unit supplies the injector with ozone gas; the injector injects the ozone gas into the water to form an ozonated fluid, wherein the injector is in fluid communication with a mixing vessel;
each of the plurality of ozonated fluid generators in fluid communication with a single fill line and an individual distribution line; and,
the system is operable to direct ozonated fluid to one or more of the individual distribution lines or to the single fill line.

15. A system for producing a high volume of ozonated fluid, comprising:
a water input line;
a flow sensor, the water input line in fluidic communication with the flow sensor;
a water line distributor, the water line distributor separates incoming water from the water input line into a first injector water line and a second injector water line;
a first ozone generation system comprising a first plurality of ozone gas generators and a first ozone supply line;
a second ozone generation system comprising a second plurality of ozone gas generators and a second ozone supply line;
a first injector; the first injector water line in fluidic communication with the first injector; the first ozone supply line in supply communication with the first injector; the first injector injects ozone gas into the water to form a first ozonated fluid, and the first injector supplies a first fill line with the first ozonated fluid;
a second injector; the second injector water line in fluidic communication with the second injector; the second ozone supply line in supply communication with the second injector; the second injector injects ozone gas into the water to form a second ozonated fluid, and the second injector supplies a second fill line with the second ozonated fluid; and,
the first fill line supplies a main fill line with the first ozonated fluid, and the second fill line supplies the main fill line with the second ozonated fluid.

16. The system according to claim 15, wherein the first fill line and the second fill line fluidly connect to a fill line reducer, wherein the fill line reducer fluidly connects to the main fill line.

17. The system according to claim 15, wherein the first ozone generation system and the second ozone generation system simultaneously generate ozone gas, and the first injector and the second injector simultaneously inject the ozone gas into the water.

18. The system according to claim 15, wherein the flow sensor is in electrical communication with the first ozone generation system and the second ozone generation system, and a flow of water through the flow sensor activates the first ozone generation system and the second ozone generation system.

19. The system according to claim 15, wherein the main fill line outputs a combination of the first ozonated fluid and the second ozonated fluid.

20. The system according to claim 15, wherein the system produces ozonated fluid at approximately ½ to approximately 10 gallons per minute at an oxidation reduction potential of approximately 700 millivolts to approximately 1000 millivolts.

21. The system according to claim 15, wherein the main fill line includes a valve or other regulating structure, and opening the valve causes the production of ozonated fluid by the system.

22. The system according to claim 15, wherein the main fill line includes a valve or other regulating structure, and opening the valve causes a flow of water through the system which activates the production of ozonated fluid by the system.

23. The system according to claim 15, wherein the system is fluidly connected to a water supply to supply the system with water, wherein water pressure from the water supply pressurizes the system.

24. A system for producing a high volume of ozonated fluid, comprising:
a water input line;
a flow sensor, the water input line in fluidic communication with the flow sensor;
a first ozonated fluid generator in fluidic communication with the water input line to supply the generator with water; wherein the first ozonated fluid generator generates a first ozonated fluid;
a second ozonated fluid generator in fluidic communication with the water input line to supply the generator with water; wherein the second ozonated fluid generator generates a second ozonated fluid;
the flow sensor in electrical communication with the first and second ozonated fluid generators; wherein the flow sensor activates the first and second ozonated fluid generators upon sensing flow of water;
the first ozonated fluid generator directs to the first ozonated fluid to a fill line; and,
the second ozonated fluid generator directs the second ozonated fluid to the fill line.
